(19) United States
(12) Patent Application Publication
(10) Pub. No.: US 2010/0137608 A1
(43) Pub. Date: Jun. 3, 2010

(54) POLY-N-HETEROCYCLIC CARBENE TRANSITION METAL COMPLEXES AND N-HETEROCYCLIC CARBENE TRANSITION METAL COMPLEXES FOR CARBON-SULFUR AND CARBON-OXYGEN COUPLING REACTIONS

(76) Inventor: Yugen Zhang, Singapore (SG)

Correspondence Address: KLARQUIST SPARKMAN, LLP, 121 SW SALMON STREET, SUITE 1600, PORTLAND, OR, 97204, US

(21) Appl. No.: 12/598,413

(22) PCT Filed: May 2, 2008
(86) PCT No.: PCT/SG08/00157

§ 371 Date: Oct. 30, 2009

Related U.S. Application Data
(60) Provisional application No. 60/924,164, filed on May 2, 2007.

(51) Int. Cl. C07F 15/04; C07C 319/00; C07C 41/01

(52) U.S. Cl. 548/103; 568/56; 568/58; 568/663

(57) ABSTRACT

Methods for carbon-sulfur (C—S) or carbon-oxygen (C—O) coupling reactions are provided. The methods involve the use of a transition metal complex comprising a heterocyclic carbene ligand complexed with a transition metal. Transition metal complexes comprising a heterocyclic carbene ligand complexed with nickel are also provided. The nickel heterocylic carbene complexes may be used for C—S or C—O coupling reactions.

This application claims the benefit of U.S. Provisional Patent Application Ser. No. 60/924,164, filed May 2, 2007, which is incorporated herein by reference in its entirety.

### FIELD

This invention relates to a poly-N-heterocyclic carbene (p-NHC) transition metal complex and a N-heterocyclic carbene (NHC) transition metal complex for carbon-sulfur (C—S) and carbon-oxygen (C—O) coupling reactions. This invention further relates to a p-NHC nickel complex and a NHC nickel complex, which may be used for C—S and C—O coupling reactions.

### BACKGROUND

Organosulfur chemistry has been receiving more and more attention since sulfur-containing groups serve an auxiliary function in organic synthetic sequences. Aryl sulfides are also a common functional group in numerous pharmaceutically active compounds. However, synthesis of aryl-sulfur bonds was still considered a challenge until the development of a series of palladium organophosphane (Pd—PR₃) catalysts, including those developed by Buchwald, Hartwig and others. (See, for example, M. Murata, S. L. Buchwald, Tetrahedron 2004, 60, 7397; and M. A. Fernandez-Rodriguez, Q. Shen, U. F. Hartwig, J. Am. Chem. Soc. 2006, 128, 2180.) However, limitations of Pd—PR₃ catalysts have been reported, including low turnover number, high cost and toxicity of the organophosphane (PR₃) ligands. The development of several other transition metal organophosphane-based catalysts has been reported. However, they have also been reported as exhibiting a number of limitations, including low activities.

For C—O coupling, as compared to C—S coupling, success with an analogous process for the addition of alcohols to produce aromatic ethers has been reported. Problems in the existing approaches for C—O coupling, involving Mitsunobu processes, copper catalysts and Pd—PR₃ catalysts, have also been reported. It has been reported that Mitsunobu processes may be complicated by the formation of by-products. Slow reaction rates and low tolerance of substrates of copper/pyridine catalysts have been reported. Palladium catalysts in C—O coupling have also been reported to exhibit the same limitations observed in C—S coupling, including low turnover numbers, and the use of expensive and toxic PR₃ ligands.

N-heterocyclic carbenes have been reported as a class of ligands which can be used for transition metal catalysis in view of their similarity to electron-rich organophosphanes, and the σ-donating properties of NHCs. Use of metal-NHC complexes in many processes, including olefin metathesis, carbon-carbon (C—C) or carbon-nitrogen (C—N) cross-coupling, olefin hydrogenation, transfer hydrogenation of ketones, and symmetric or asymmetric hydrosilylation, have been reported.

The development of several types of supported transition metal-NHC complexes to exploit the benefits of heterogeneous catalysts, including resin-supported Pd-NHC complexes for Heck reaction, has been reported. The development of metal-NHC complexes supported on mesoporous materials and particles/polymer hybrid materials for various reactions has been reported. However, limitations of the catalysts supported on polymeric or mesoporous materials have been reported, including low activity, multi-step syntheses, low catalyst loading and others issues.

The development of a class of heterogeneous NHC catalysts, main chain p-NHCs, which spontaneously form nanometer- or micron-sized colloidal particles, has been reported (WO 2007/114,793). Poly-imidazolium salts or p-NHC particles were reported to be insoluble in common solvents, and used as heterogeneous catalysts or solid ligands for catalysis. The synthesis of p-NHC metal complexes from the poly-imidazolium salt, and the catalytic properties of Pd-p-NHCs in heterogeneous Suzuki coupling reactions have been reported (WO 2007/114,793), p-NHC is a polymer material with free carbene units in its main chain, and has been reported to be easy to synthesize. p-NHC has also been reported as having versatile properties in coordination with different transition metals and can support metals to generate heterogeneous organometallic catalysts.

It has been reported that Ni-NHC complexes demonstrated efficient carbon-fluorine and carbon-carbon bond activation. Ni-NHC catalyzed hydrothiolation of alkynes has also been reported. Ni complexes have been reported to catalyze C—S coupling. However, it has been reported that good activities were only achieved with aryl iodides.

Organophosphane-free catalysts for C—S and C—O coupling reactions are desired.

### SUMMARY

In one broad aspect of the invention, there is provided a method for carbon-sulfur (C—S) or carbon-oxygen (C—O) coupling comprising: a) mixing, in any order, a thiol-containing compound, an aryl halide and a transition metal complex to obtain C—S coupling; or b) mixing, in any order, an alkoxide or aryloxide, an aryl halide and a transition metal complex to obtain C—O coupling, wherein the transition metal complex comprises a heterocyclic carbene ligand complexed with a transition metal other than palladium.

In another broad aspect of the invention, there is provided a method for carbon-sulfur (C—S) or carbon-oxygen (C—O) coupling comprising: a) mixing, in any order, a thiol-containing compound, an aryl halide and a transition metal complex to obtain C—S coupling; or b) mixing, in any order, an alkoxide or aryloxide, an aryl halide and a transition metal complex to obtain C—O coupling, wherein the transition metal complex comprises a heterocyclic carbene ligand complexed with nickel.

In a further broad aspect of the invention, there is provided a transition metal complex comprising a poly-N-heterocyclic carbene complexed with nickel.

In still another broad aspect of the invention, there is provided a transition metal complex comprising a N-heterocylic carbene complexed with nickel.

### BRIEF DESCRIPTION OF THE DRAWINGS

Embodiments of the invention will be discussed with reference to the following Figures:

FIG. 1 displays the structures of a poly-imidazolium salt 1, a poly-imidazolidene carbene 2 and a poly-imidazolidene carbene metal complex 3.

FIG. 2 displays synthesis of a Ni-p-NHC catalyst B from a p-NHC A.

### DETAILED DESCRIPTION

The present invention relates to methods for C—S and C—O coupling using a transition metal complex.

In an embodiment of the invention, the transition metal complex may comprise, for example, and without limitation, heterocyclic groups. For example, and without limitation, the transition metal complex may comprise a heterocylic carbene ligand complexed with a transition metal.

In an embodiment, the heterocyclic carbene ligand may be, for example, and without limitation, a poly-N-heterocyclic carbene. For example, and without limitation, the transition metal complex may comprise one or more monomer units comprising two heterocyclic groups joined by a linker group.

In an embodiment, the transition metal complex may comprise, for example, and without limitation, one or more monomer units represented by the formula (I).

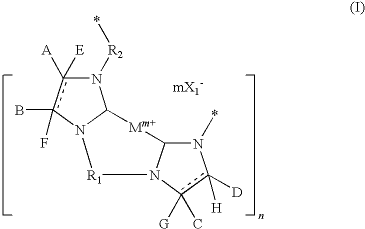

In formula (I), each of R₁ and R₂ is a linker group. Each of R₁ and R₂ may be independently a rigid linker group, a non-rigid linker group or a semi-rigid linker group. R₁ and R₂ may be the same or different.

Suitable rigid linker groups would be understood to and can be determined by those of ordinary skill in the art, and may include, for example, and without limitation, aromatic groups, heteroaromatic groups, cycloaliphatic groups, suitably rigid alkenes and suitably rigid alkynes. Suitable rigid linker groups may include, for example, optionally substituted ethenyl (e.g. ethenediyl, propen-1,2-diyl, 2-butene-2,3-diyl, etc.), ethynyl (e.g. ethynediyl, propynediyl, but-2,3-yne-1,4-diyl, etc.), aryl (1,3-phenylene, 1,4-phenylene, 1,3-naphthylene, 1,4-naphthylene, 1,5-naphthylene, 1,6-naphthylene, 1,7-naphthylene, 1,8-naphthylene, etc.), heteroaryl (e.g. 2,6-pyridinediyl, 2,6-pyrandiyl, 2,5-pyrrolediyl, etc.), and cycloalkyl (e.g. 1,3-cyclohexanediyl, 1,4-cyclohexanediyl, 1,3-cyclopentanediyl, 1,3-cyclobutanediyl, etc.) linker groups.

Suitable non-rigid and semi-rigid linker groups would be understood to and can be determined by those of ordinary skill in the art, and may include, for example, and without limitation, an alkyl, alkenyl (other than ethenyl), alkylaryl and other suitable linker groups. Suitable non-rigid or semi-rigid linker groups may include, for example, —(CH₂)ᵤ—, where u is between 1 and about 10, and which non-rigid or semi-rigid linker groups may be optionally substituted and/or branched (e.g. 1,2-ethanediyl, 1,2- or 1,3-propanediyl, 1,2-, 1,3-, 1,4- or 2,3-butanediyl, 2-methyl-butane-3,4-diyl, etc.).

The linker groups may be optionally substituted (e.g. by an alkyl group, an aryl group, a halide or some other substituent) or may comprise a heteroatom such as O, S, N (e.g. R₁ or R₂ may independently be —CH₂OCH₂—, —CH₂OCH₂CH₂—, —CH₂OCH(CH₃)—, —(CH₂OCH₂)ₚ— (where p is between 1 and about 100), —CH₂NHCH₂—, CH₂N(CH₃)CH₂, —CH₂N(Ph)CH₂—, —CH₂SCH₂—, etc.). The heteroatom may be disposed so that it is also capable of complexing or bonding to the transition metal.

In an embodiment, for example, and without limitation, R₁ may be a rigid linker group and R₂ may be a non-rigid or semi-rigid linker group.

In formula (I), M is a transition metal and the symbol * indicates an end of the monomer unit.

In formula (I), X₁⁻ is a counterion. In an embodiment, X₁⁻ may be, for example, and without limitation, a halide, such as, for example, bromide, chloride or iodide. Other suitable X₁⁻ may be, for example, acetate, nitrate, trifluoroacetate, etc. In an embodiment, X₁⁻ may be coordinated with the transition metal.

The formulae described throughout this entire specification representing the monomer unit(s) of the transition metal complex may be represented with a m+charge on M as shown above, or the formulae may be represented as having bonds linking the X₁⁻s to M. Those of ordinary skill in the art will appreciate that the transition metal M may be doubly coordinated as represented in the formula above, or the transition metal M may be coordinated differently, for example, and without limitation, the transition metal M may be singly or triply coordinated. Thus, the transition metal may be M^{m+}, where m is an integer of 1, 2, 3, 4, 5, 6 or 7, although typically m will be 1, 2 or 3. The number of X₁⁻ groups will then generally be mX₁⁻ groups, where m is defined as above. While each X₁⁻ might be the same or different, generally each X₁⁻ is selected to be the same counterion.

In formula (I), n is the degree of polymerisation. In an embodiment, n may be, for example, and without limitation, a value where the transition metal complex is insoluble in solvents used for the coupling reactions. n may be, for example, and without limitation, greater than about 5, or greater than about 10, 15, 20, 30, 40, 50, 60, 70, 80, 90, 100, 200, 300, 400, 500, 600, 700, 800, 900 or 1000, or may be between about 5 and 1000, 10 and 1000, 50 and 1000, 100 and 1000, 200 and 1000, 500 and 1000, 5 and 500, 5 and 200, 5 and 100, 5 and 50, 5 and 20, 5 and 10, 10 and 50, 50 and 500, 50 and 200, 50 and 100 or 100 and 300, and including any specific value within these ranges, such as, for example, and without limitation, about 5, 6, 7, 8, 9, 10, 15, 20, 30, 40, 50, 60, 70, 80, 90, 100, 150, 200, 250, 300, 350, 400, 450, 500, 600, 700, 800, 900 or 1000.

In formula (I), [symbol] represents a single bond or a double bond, wherein when [symbol] represents a double bond, E, F, G and H are not present. In an embodiment, each of A, B, C and D, and, if present, E, F, G and H may independently be, for example, and without limitation, hydrogen or a substituent which is not hydrogen. Each of A, B, C, D, E, F, G and H may independently be, for example and without limitation, hydrogen, alkyl (e.g. straight chain, branched chain, cycloalkyl, etc.), aryl (e.g. phenyl, naphthyl, etc.), halide (e.g. bromo, chloro, etc.), heteroaryl (e.g pyridyl, pyrrolyl, furanyl, furanylmethyl, thiofuranyl, imidazolyl, etc.), alkenyl (e.g. ethenyl, 1-, or 2-propenyl, etc.), alkynyl (e.g. ethynyl, 1- or 3-propynyl, 1-, 3- or 4-but-1-ynyl, 1- or 4-but-2-ynyl, etc.) or some other substituent. A, B, C and D and, if present, E, F, G and H, may be all the same, or some or all may be different.

The alkyl group may have, for example, and without limitation, between about 1 and 20 carbon atoms (provided that cyclic or branched alkyl groups have at least 3 carbon atoms), or between about 1 and 12, 1 and 10, 1 and 6, 1 and 3, 3 and 20, 6 and 20, 12 and 20, 3 and 12 or 3 and 6, including any specific number within these ranges. For example, and without limitation, the alkyl group may be, methyl, ethyl, 1- or 2-propyl, isopropyl, 1- or 2-butyl, isobutyl, tert-butyl, cyclopentyl, cyclopentylmethyl, cyclohexyl, cyclohexylmethyl, methylcyclohexyl, etc.

The substituents may be optionally substituted (e.g. by an alkyl group, an aryl group, a halide or some other substituent) or may comprise a heteroatom such as O, S, N (e.g. the substituent may be methoxymethyl, methoxyethyl, ethoxymethyl, polyoxyethyl, thiomethoxymethyl, methylaminomethyl, dimethylaminomethyl, etc.).

Each of A, B, C and D, and, if present, E, F, G and H may independently be chiral or achiral.

In an embodiment, for example, and without limitation, any two of A, B, C and D, and, if present, E, F, G and H may be joined to form a cyclic structure. In an embodiment, at least one heterocyclic ring of formula (I) may have fused or spiro-joined rings. For example, and without limitation, when [symbol] represents a single bond, any pair of substituents A, B, C, D, E, F, G and H attached to the same carbon atom may be joined to form, for example, a cyclopentyl, cyclohexyl or some other ring. For example, where A and E form a cyclopentyl ring, a 1,3-diazaspiro[4.4]nonane structure may be formed. In an embodiment, for example, and without limitation, any pair of substituents A, B, C, D, E, F, G and H attached to adjacent carbon atoms may be joined to form, for example, a cyclopentyl, cyclohexyl or some other ring. For example, where A and B form a cyclopentyl ring, a 1,3-diazabicyclo[3.3.0]octane structure may be formed.

In an embodiment, when [symbol] represents a single bond, any pair of substituents A, B, C, D, E, F, G and H attached to the same carbon atom may represent a single substituent attached to the carbon atom by a double bond. In an embodiment, the monomer unit(s) may be represented by, for example, and without limitation, the formula (Ia), (Ib) or (Ic):

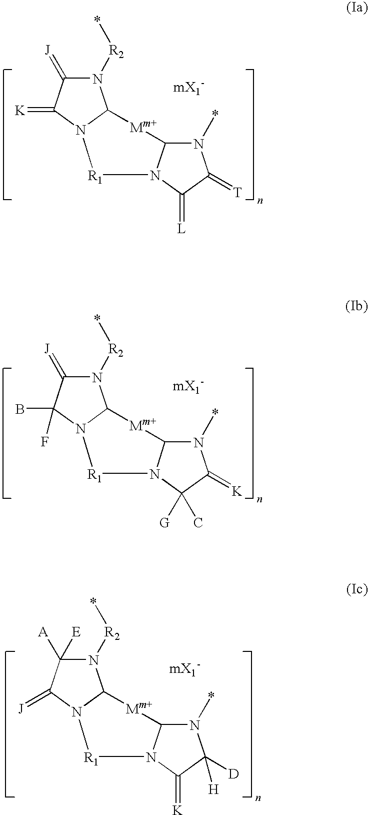

wherein each of R₁, R₂, M, *, X₁⁻, A, B, C, D, E, F, G, H, m and n may be defined as anywhere above, and each of J, K, L and T may independently be, for example, and without limitation, ═CPQ or ═NP, where P and Q may independently be, for example, and without limitation, hydrogen or a substituent which is not hydrogen including those defined for A to H above. J, K, L and T may independently be, for example, ═CH₂, ═CHCH₃, ═CHPh, ═NCH₃ or ═NPh, or some other suitable double bonded group.

In an embodiment, when [symbol] represents a double, at least one heterocyclic ring of formula (I), may be, for example, and without limitation, fused with an aromatic or heteroaromatic ring. In an embodiment, the monomer unit(s) may be represented by, for example, and without limitation, the formula (II).

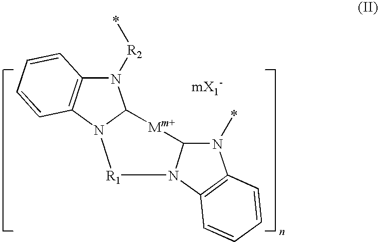

wherein each of R₁, R₂, M, *, X₁⁻, m and n may be defined as anywhere above.

In an embodiment of the invention, the heterocyclic carbene ligand may be, for example, and without limitation, a N-heterocyclic carbene copolymer. For example, and without limitation, the copolymer may comprise two or more different monomer units. In an embodiment, one, some or all of the different monomer units may be represented by the formulae as described anywhere above. In an embodiment, the copolymer may be an alternating copolymer.

In an embodiment of the invention, the transition metal complex may be, for example, nickel poly-imidazolidene (Ni-pIm) or nickel poly-benzoimidazolidene (Ni-pBIm).

In an embodiment, the carbene centres of the p-NHC as described anywhere above, may be in the main chain of the polymer.

In an embodiment, the transition metal complex may be, for example, and without limitation, in the form of one or more particles. The transition metal complex may be, for example, in the form of amorphous particles, spherical particles or microcrystalline particles. The particles may be, for example, and without limitation, colloidal particles. The particles may be, for example, and without limitation, micron-sized or nanometer-sized colloidal particles. The particles may be, for example, and without limitation, between about 100 nm to about 10 microns in diameter. The particles may have, for example, and without limitation, a diameter between about 100 nm and 1 micron, 100 and 500 nm, 500 nm and 10 microns, 1 and 10 microns, or 100 nm and 1 micron, and including any specific value within these ranges, such as, for example, and without limitation, about 100, 200, 300, 400, 500, 600, 700, 800 or 900 nm, or about 1, 1.5, 2, 2.5, 3, 3.5, 4, 4.5, 5, 6, 7, 8, 9 or 10 microns. Those of ordinary skill in the art will appreciate that the size and shape of the particles may depend on the nature of the monomer unit(s) used, and the conditions of synthesis of the polymer, particularly the solvent used in the polymerisation process.

In an embodiment of the invention, the heterocyclic carbene ligand may be, for example, and without limitation, a N-heterocyclic carbene. In an embodiment of the invention, the NHC ligand of the transition metal complex may be represented by, for example, and without limitation, the formula (III).

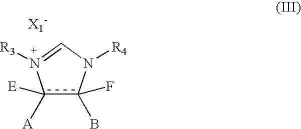

In formula (III), each of X₁⁻, A, B and, if present, E and F may be defined as anywhere above. Each of R₃ and R₄ represents a substituent which is not hydrogen including those defined for A to H anywhere above. [symbol] represents a single bond or a double bond, wherein when [symbol] represents a double bond, E and F are not present. In an embodiment, any two of A, B and, if present, E and F may be joined to form a cyclic structure including those described for formula (I) above. In an embodiment, when [symbol] represents a single bond, any pair of substituents A, B, E and F attached to the same carbon atom may represent a single substituent attached to the carbon atom by a double bond. In an embodiment, the NHC ligand may be represented by, for example, and without limitation, the formula (IIIa), (IIIb) or (IIIc):

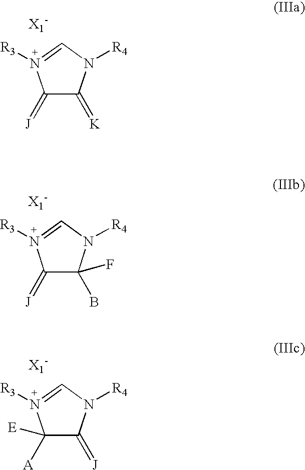

wherein each of X₁⁻, A, B, B, F, R₃, R₄, J and K may be as defined anywhere above.

In an embodiment, when [symbol] is a double bond, the heterocyclic ring of formula (III) may be, for example, and without limitation, fused with an aromatic or heteroaromatic ring. In an embodiment, the NEC ligand may be represented by, for example, and without limitation, the formula (IV):

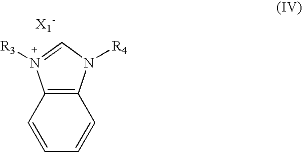

wherein X₁⁻, R₃ and R₄ are as defined anywhere above.

In an embodiment of the invention, the NHC ligand may be, for example, and without limitation, a bridged bidentate ligand. In an embodiment, the NHC ligand may be represented by, for example, and without limitation, the formula (V) or (VI):

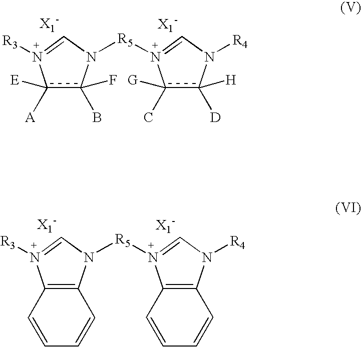

wherein X₁⁻, R₃, R₄, [symbol] , A, B, C, D and, if present, E, F, G and H may be described as anywhere above. R₅ may be, for example, and without limitation, a linker group including those described for R₁ and R₂ above.

In an embodiment, the transition metal complex may have, for example, and without limitation, a NHC ligand/transition metal ratio of from 1 to 5 including any specific value within this range, such as, for example, and without limitation, 1, 2, or 3. In an embodiment, the NHC ligand/transition metal ratio may be, for example, 2.

Transition metals are understood as falling within Groups IIIB, IVB, VB, VIIB, VIIB, VIIIB, IB and IIB in the Periodic Table of the Elements. In an embodiment of the invention, the transition metal of the transition metal complex may be, for example, and without limitation, a transition metal capable of complexing with one, two or three carbene (—C:—) centres, and also optionally with a heteroatom, wherein the transition metal is not palladium. In one embodiment, the transition metal may be, for example, and without limitation, a Group VIIIB metal. In an exemplary embodiment, the transition metal may be, for example, nickel.

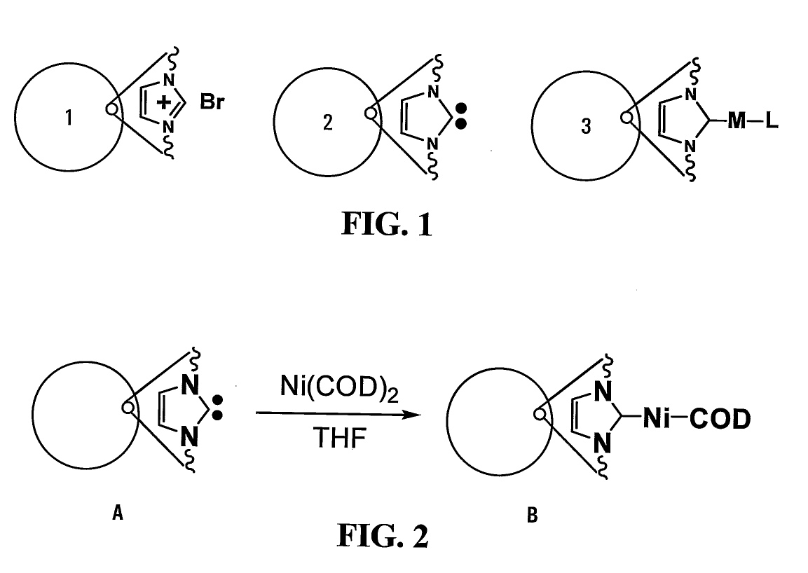

The transition metal complexes may be prepared from the corresponding free heterocyclic carbenes and/or the corresponding heterocyclic salts (see, for example, WO 2007/114,793). By way of example only, and without limitation, a poly-imidazolium salt 1, a free poly-imidazolidene carbene 2 and a poly-imidazolidene carbene metal complex 3 are shown in FIG. 1, wherein M represents a transition metal as described anywhere above and L represents a ligand, including, for example, and without limitation, cyclooctadiene (COD). For example, and without limitation, FIG. 2 shows the synthesis of nickel poly-imidazolidene (Ni-pIm) catalyst B from poly-imidazolidene free carbene polymer particles A and Ni(COD)₂.

The transition metal complexes as described anywhere above may be used to catalyse C—S or C—O coupling reactions.

In an embodiment of the invention, the C—S coupling reaction may involve an aryl halide substrate and a thiol-containing compound.

Suitable aryl halides for the C—S coupling reactions would be understood to or can be determined by those of ordinary skill in the art, and may include, for example, and without limitation, aryl iodides, aryl bromides and aryl chlorides. The aryl group of the aryl halide may be optionally substituted with a substituent which is not hydrogen including those defined for A to H anywhere above. The aryl group of the aryl halide may be fused with an aromatic or heterocyclic ring. In an embodiment of the invention, the aryl halide may be activated, non-activated or deactivated. Suitable thiol-containing compounds for the C—S coupling reactions would be understood to or can be determined by those of ordinary skill in the art, and may include, for example, and without limitation, aryl thiols and alkyl thiols. The aryl and alkyl moieties of the aryl and alkyl thiols may include the aryl and alkyl groups as defined anywhere above.

An embodiment of the present invention may be represented by, for example, and without limitation, the following scheme:

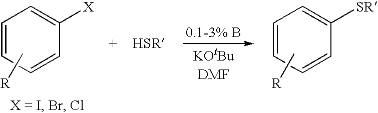

wherein B represents Ni-pIm, R of the aryl halide represents hydrogen or a substituent which is not hydrogen as described anywhere above, and R′ of the thiol represents aryl or alkyl as described anywhere above.

The mechanism of Pd—PR₃ catalysts in coupling reactions has been well studied. By way of example, and without limitation and without being bound by theory, it is believed that Ni-NHC catalysts are undergoing the same oxidative addition and reductive elimination cycle, as represented in the following scheme:

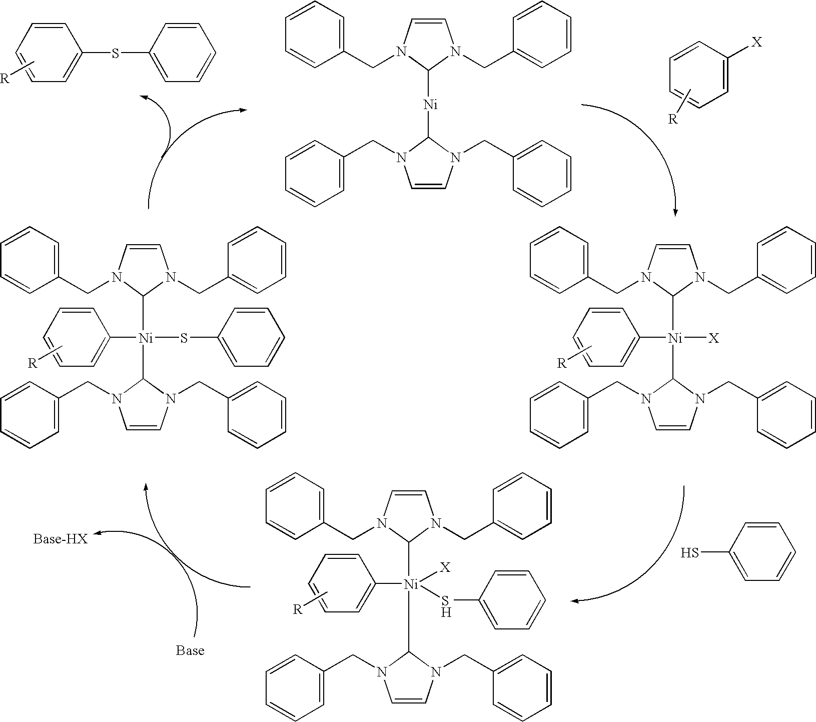

wherein R and X are as defined anywhere above. While sterically hindered ligands are generally good in the reductive elimination step they would generally slow down the oxidative addition process. On the other hand, strong electron-donating ligands may help the oxidative addition of aryl halides but are generally not good in reductive elimination. It is believed that tuning the steric hindrance and electron-donating properties of ligands may be a consideration in catalyst development.

In an embodiment of the invention, the C—O coupling reaction may involve an aryl halide and an alkoxide or aryloxide.

Suitable aryl halides for the C—O coupling reactions would be understood to or can be determined by those of ordinary skill in the art, and may include those aryl halides defined for the C—S coupling above. Suitable alkoxides and aryloxides for the C—O coupling reactions would be understood to and can be determined by those of ordinary skill in the art. The alkyl and aryl moieties of the alkoxides and aryloxides may include the alkyl and aryl groups as defined anywhere above. Suitable alkoxides may include, for example, and without limitation, primary, secondary and tertiary alkoxides. The alkoxides and aryloxides may be substituted with a substituent which is not hydrogen including those defined for A to H anywhere above.

An embodiment of the present invention may be represented by, for example, and without limitation, the following scheme:

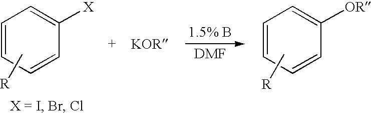

wherein B represents Ni-pIm, R of the aryl halide is as defined anywhere above and R″ represents an alkyl or aryl group as defined anywhere above.

The transition metal complex, the thiol-containing compound and the aryl halide for the C—S coupling, or the transition metal complex, the alkoxide or aryloxide and the aryl halide for the C—O coupling may be mixed in any order. For the C—S coupling, for example, and without limitation, the transition metal complex may be first mixed with any one of the thiol-containing compound and the aryl halide, or the thiol-containing compound and the aryl halide may be first mixed together before mixing with the transition metal complex. For the C—O coupling, for example, and without limitation, the transition metal complex may be first mixed with any one of the alkoxide or aryloxide and the aryl halide, or the alkoxide or aryloxide and the aryl halide may be first mixed together before mixing with the transition metal complex.

The reaction conditions of the C—S and C—O coupling reactions would be understood to and can be determined by those of ordinary skill in the art. The coupling reactions may be carried out in the presence of a solvent. Suitable solvents would be understood to and can be determined by those of ordinary skill in the art, and may include, for example, and without limitation, N,N-dimethylformamide tetrahydrofuran (THF) or toluene. In an embodiment, the transition metal complex may be insoluble in the solvent, i.e. the transition metal complex may function as a heterogeneous catalyst. In an embodiment, the transition metal complex may be soluble or at least partially soluble in the solvent, i.e. the transition metal complex may function as a homogeneous catalyst. Suitable reaction temperatures would be understood to and can be determined by those of ordinary skill in the art, and may include, for example, and without limitation, from about 80 to 120° C., and including any specific value within this range, such as, for example, 100 or 110° C.

The amount of transition metal complex used would be understood to and can be determined by those of ordinary skill in the art, and may include from less than about 5 mol %, between about 0.1 to 3 mol %, and including any specific value within these ranges, for example, 0.1 mol %, 1.5 mol %, 3 mol % or 4 mol %. Suitable amounts of the aryl halide and thiol-containing compound in the C—S coupling reactions and the aryl halide and aryloxide or alkyloxide in the C—O coupling reactions would be understood to and can be determined by those of ordinary skill in the art. For example, and without limitation, the coupling reagents may be used in accordance with their stoichiometric ratios.

In an embodiment, the C—S coupling reaction may be carried out in the presence of a suitable base. For example, and without limitation, suitable bases may include KO^{t}Bu, Cs₂CO₃, Na₂CO₃ and NaO^{t}Bu.

In an embodiment, the transition metal complex may be recycled to catalyse one or more subsequent reactions.

Those of ordinary skill in the art will appreciate that the method may optionally comprise separating the product from the reaction mixture, for example, and without limitation, by filtration, chromatographic separation, recrystallization or other suitable separation processes.

### EXAMPLES

All solvents were used as obtained from commercial suppliers, unless otherwise noted. Centrifugation was performed on Eppendorf™ Centrifuge 5810R (4000 rpm, 10 min). Gas liquid chromatography was performed on Agilent™ 6890N Series gas chromatograph equipped with a split-mode capillary injection system and flame ionization detector. Gas chromatography-mass spectrometry (GC-MS) was performed on Shimadzu™ GCMS 02010. Inductively coupled plasma mass spectrometry (ICP-MS) was performed on ELAN™ 9000/DRC system. Progress of the catalytic reactions was typically monitored by GC or GC-MS analysis of reaction aliquots.

Synthesis of Ni(0)-p-NHC Catalyst

82.5 mg of Ni(COD)₂ (COD=cyclooctadiene) (0.3 mmol) were added to a suspension of poly-imidazolidene (p-Im) A (1 g) in THF in the glove box. The mixture was stirred for 16 h at room temperature. The suspension was then filtered, and washed with DMF (10 ml), THF (10 ml×2) and ether (10 ml). The nickel poly-imidazolidene (Ni-pIm) catalyst B was dried in vacuum, and collected as a yellow powder. The nickel loading on polymer (0.3 mmol/g) was confirmed by ICP-MS. Nickel poly-benzoimidazolidene (Ni-pBIm) D (0.3 mmol/g) was prepared from poly-benzoimidazolidene (pBIm) C by using the same procedure as the synthesis of Ni-pIm B.

### C—S Coupling Reactions Over Ni-p-NHC Catalysts

All reactions were carried out in inert atmosphere. Ni-pIm B (10 mg, 0.003 mmol of Ni), KO^{t}Bu (0.25 mmol), thiophenol (0.22 mmol), 4-chlorobenzenetrifluoride (0.2 mmol) were mixed with 2 ml of DMF in a reaction vial. The vial was capped, and the reaction mixture was stirred at 100° C. for 16 h. After completion of the reaction, the reaction mixture was centrifuged, and the solution was removed. This procedure was repeated at least three times by using dry DMF as the washing solvent. The combined liquid was collected for yield measurement. The recovered catalyst was used directly for the next run.

### C—O Coupling Reactions Over Ni-p-NHC Catalysts

Ni-pIm B (10 mg, 0.003 mmol of Ni), KO^{t}Bu (0.25 mmol), 4-chlorobenzenetrifluoride (0.2 mmol) were mixed with 2 ml of DMF in a reaction vial. The vial was capped, and the reaction mixture was stirred at 100° C. for 16 h. After completion of the reaction, the reaction mixture was centrifuged, and the solution was removed. This procedure was repeated at least thrice using dry DMF as the washing solvent. The combined liquid was collected for yield measurement. The recovered catalyst was used directly for the next run.

The catalytic activity of Ni-pIm catalyst B was investigated in C—S coupling of aryl halides. Several solvents and bases were examined for the reaction of 4-chlorobenzotrifluoride and thiophenol over Ni-pIm catalyst B (1.5 mol %). Sulfide products were obtained in excellent yields (94%) in DMF/potassium tert-butoxide (KO^{t}Bu) system, but moderate or low yields were obtained in other solvents (toluene or THF).

Conversion of both activated and non-activated aryl halides to the corresponding sulfides was generally observed with good to excellent yields. However, only moderate or low yields were typically observed for deactivated aryl bromides and chlorides. Yields above 95% are considered excellent yields, yields from 80 to 95% are considered good yields, yields from 50 to 80% are considered moderate yields and yields less than 50% are considered low yields. Results from experiments conducted are presented in Table 1.

TABLE 1
C—S coupling reactions over Ni-pIm catalyst B.^{[a]}

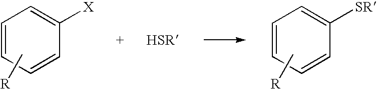

| Entry | X | B [mol %] | R | Product | Yield [%]^{[b]} |
|---|---|---|---|---|---|
| 1^{[g]} | I | 1.5 | H | 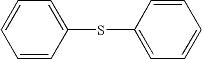 | 99 |
| 2^{[f]} | I | 1.5 | OMe | 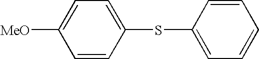 | 99 |
| 3^{[h]} | Br | 1.5 | CF₃ | 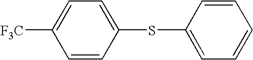 | 99 |
| 4^{[h]} | Br | 1.5 | COMe | 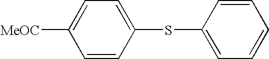 | 99 |
| 5^{[c],[g]} | Br | 1.5 | H | 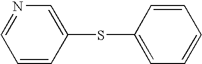 | 99 |
| 6^{[f]} | Br | 1.5 | Me | 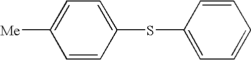 | 65 |
| 7^{[f]} | Br | 1.5 | OMe | 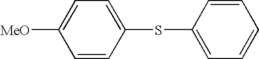 | 51 |
| 8^{[h]} | Cl | 1.5 | CF₃ | 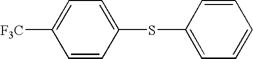 | 94 |
| 9^{[d],[h]} | Cl | 1.5 | CF₃ | 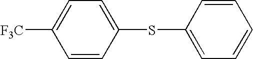 | 94 |
| 10^{[h]} | Cl | 1.5 | COMe | 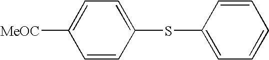 | 99 |
| 11^{[e],[h]} | Cl | 1.5 | CF₃ | 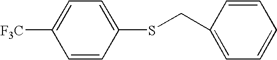 | 99 |
| 12^{[e],[h]} | Cl | 1.5 | CF₃ | 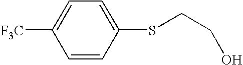 | 99 |

^{[a]}Reaction conditions: 0.2 mmol of aryl halides, 0.22 mmol of thiols in 2 ml of DMF, 100° C., 16 h.
^{[b]}GC yields.
^{[c]}3-Bromopyridine was used as the substrate.
^{[d]}Recycled catalyst.
^{[e]}Reaction was conducted at 80° C. for 4 h.
^{[f]}Deactivated aryl halide was used as the substrate.
^{[g]}Non-activated aryl halide was used as the substrate.
^{[h]}Activated aryl halide was used as the substrate.

The C—S coupling reaction of various aryl iodides, bromides and chlorides with thiophenol was examined over Ni-pIm catalyst B (Table 1). High activities of the catalyst for aryl iodides, bromides and chlorides were observed in these experiments. The catalyst was observed to be tolerant of different functional groups on aryl halides. In addition to aryl thiols, alkyl thiols were also tested over catalyst B. Similar activities of catalyst B towards alkyl thiols and towards aryl thiols were observed.

The Ni-pIm catalyst also demonstrated excellent reusability. No deactivation was observed for the recycled catalyst (see Table 1). The Ni-pIm catalyst was observed to maintain excellent catalytic activity over multiple runs.

Comparable activities of catalyst B to most homogeneous Pd—PR₃ catalysts were observed. Catalyst B was observed to provide C—S coupling activity similar to or lower than the expensive homogeneous Pd(dba)₂/CyPF-t-Bu catalyst developed by Hartwig. Similar activities as catalyst B in the C—S coupling reactions were observed for catalyst D.

TABLE 2
C—O coupling reactions over Ni-pIm catalyst B^{[a]}

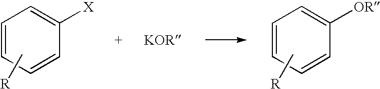

| | | B | | | Yield |
|---|---|---|---|---|---|
| Entry | X | [mol %] | R | Product | [%]^{[b]} |
| 1^{[c]} | Cl | 1.5 | CF₃ | 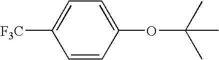 | 99 |
| 2^{[c]} | Cl | 1.5 | CF₃ | 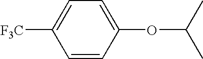 | 60 |
| 3^{[c]} | Cl | 1.5 | CF₃ | 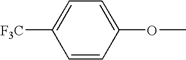 | 83 |

^{[a]}Reaction conditions: 0.2 mmol of aryl halides, 0.22 mmol of alkoxides in 2 ml of DMF, 100° C., 16 h.
^{[b]}GC yields.
^{[c]}Activated aryl halide was used as the substrate.

Direct coupling of aryl halides with alkoxides and aryloxides was investigated by using Ni-pIm catalyst B using similar reaction conditions as C—S coupling.

High activities of Ni-pIm catalyst B towards coupling aryl halides with all primary, secondary and tertiary alkoxides to form the associated esters were observed (Table 2). Activities are considered relative to other comparative catalysts. Good yields with less than 1% catalyst loading is considered as high activity.

Low conversions were observed for the coupling of aryl halides with aryloxides, and for the coupling of deactivated aryl chlorides or bromides with alkoxides. The activity of Ni-pIm catalyst B towards alkoxides was observed to be comparable with Buchwald's Pd—PR₂ catalysts (see, for example, A. V. Vorogushin, X. Huang, S. L. Buchwald, J. Am. Chem. Soc. 2005, 127, 8146).

### Synthesis of Ni(0)-NHC Catalysts

Nickel 1,3-dibenzylimidazolidene ((c)₂-Ni(0)) catalyst was synthesized by adding 82.5 mg of Ni(COD)₂ (0.3 mmol) in a glovebox to a mixture of 195 mg of c (0.6 mmol) and 68 mg of KO^{t}Bu (0.6 mmol) in 10 mL of DMF. The mixture was stirred for 1 h at room temperature, and used as the catalyst stock solution for catalytic reactions.

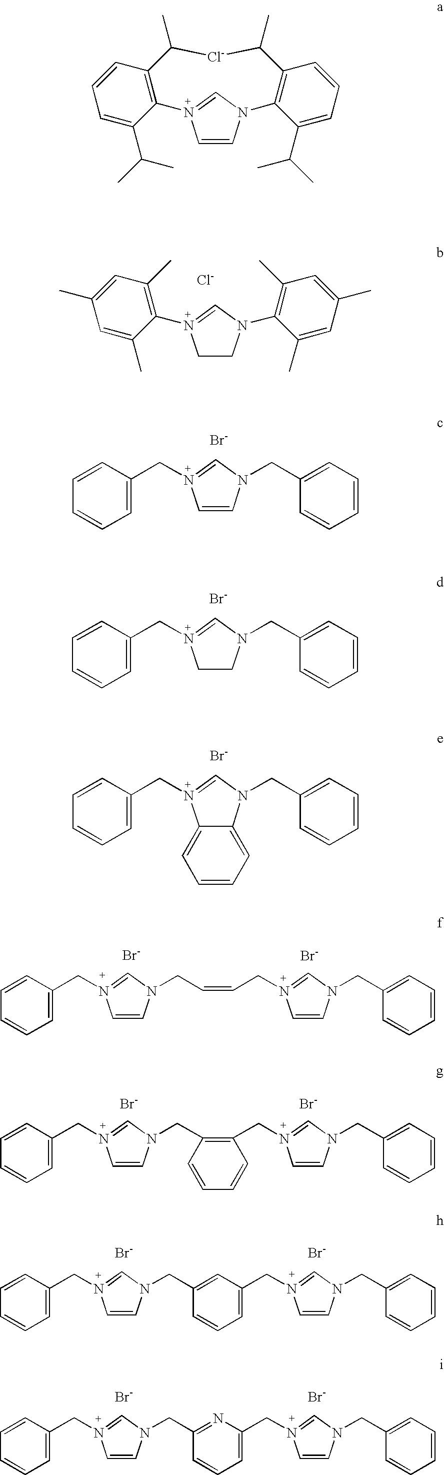

### CS Coupling Reactions Over Ni-NHC Catalysts

All reactions were performed in inert atmosphere. (c)₂-Ni solution (1 mL, 0.03 mmol of Ni), KO^{t}Su (125 mg, 1.1 mmol), thiophenol (1.05 mmol), and 4-bromotoluene (1 mmol) were mixed with 3 mL of DMF in a reaction vial. The vial was capped, and the reaction mixture was stirred at 110° C. for 16 h. Yields were measured by gas liquid chromatography (GLC) and isolation of pure product. Products were confirmed by gas chromatographymass spectrometry (GC-MS) and nuclear magnetic resonance (NMR).

### C—O Coupling Reactions Over Ni-NHC Catalysts

C—O coupling reactions over Ni-NHC catalysts were performed using similar procedures as those used for the C—O coupling reactions over Ni-p-NHC catalysts. For reaction conditions see Table 6.

TABLE 3
C—S Coupling Reactions over Ni—NHC Catalysts^{[a]}

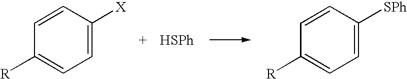

| | ligand | | Ni | | | |
|---|---|---|---|---|---|---|
| | (ligand/Ni | | catalyst | | | % |
| Entry | ratio) | X | [mol %] | R | Product | yield^{[b]} |
| 1 | a(1) | Br | 3 | Me | 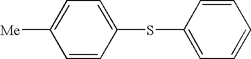 | 14^{[c]} |
| 2 | b(2) | Br | 3 | Me | 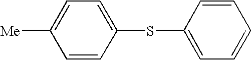 | 54^{[c]} |
| 3 | c(1) | Br | 3 | Me | 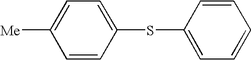 | 56^{[c]} |
| 4 | c(2) | Br | 3 | Me | 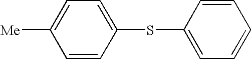 | 89^{[c]} |
| 5 | c(2) | Br | 1.5 | Me | 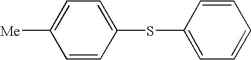 | 56^{[c]} |
| 6 | c(3) | Br | 3 | Me | 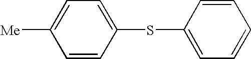 | 34^{[c]} |
| 7 | d(2) | Br | 3 | Me | 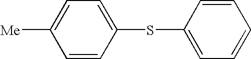 | 65^{[c]} |
| 8 | e(2) | Br | 3 | Me | 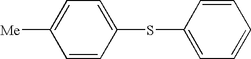 | 52^{[c]} |
| 9 | f(1) | Br | 3 | Me | 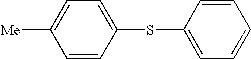 | 88^{[c]} |
| 10 | f(1) | Br | 1.5 | Me | 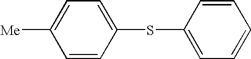 | 65^{[c]} |
| 11 | g(1) | Br | 3 | Me | 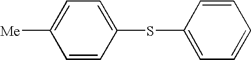 | 92^{[c]} |
| 12 | g(1) | Br | 1.5 | Me | 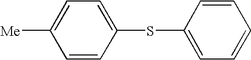 | 78^{[c]} |
| 13 | h(1) | Br | 3 | Me | 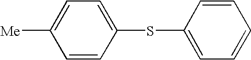 | 92^{[c]} |
| 14 | h(1) | Br | 1.5 | Me | 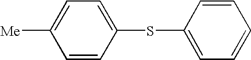 | 71^{[c]} |
| 15 | h(1) + c(1) | Br | 3 | Me | 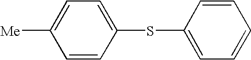 | —^{[c]} |
| 16 | h(1) + c(1) | Br | 1.5 | Me | 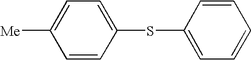 | 37^{[c]} |
| 17 | i(1) | Br | 3 | Me | 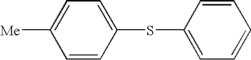 | 92^{[c]} |
| 18 | i(1) | Br | 1.5 | Me | 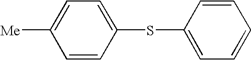 | 68^{[c]} |
| 19 | a(1) | Cl | 1 | CF₃ | 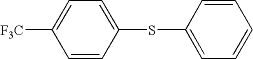 | 80^{[d]} |
| 20 | a(1) | Br | 1.5 | Me | 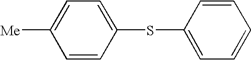 | 13.8^{[d]} |
| 21 | c(1) | Cl | 1 | CF₃ | 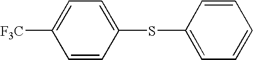 | 81^{[d]} |
| 22 | c(1) | Br | 1.5 | Me | 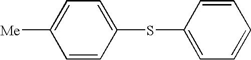 | 59^{[d]} |
| 23 | c(2) | Cl | 1 | CF₃ | 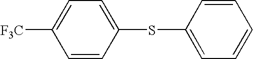 | 80^{[d]} |
| 24 | c(2) | Br | 1.5 | Me | 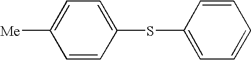 | 89^{[d]} |
| 25 | c(2) | Br | 3 | OMe | 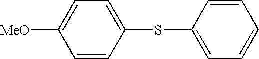 | 92^{[d]} |
| 26 | e(2) | Br | 1.5 | Me | 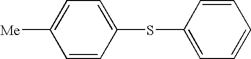 | 52^{[d]} |
| 27 | e(2) | Cl | 0.1 | CN | 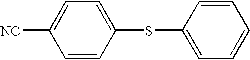 | 99^{[d]} |
| 28 | e(2) | Cl | 0.1 | CF₃ | 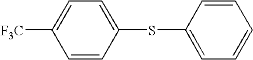 | 77^{[d]} |

^{[a]}Unless otherwise specified, the reaction conditions are 0.2 mmol of aryl halides, 0.22 mmol of thiols and Ni catalyst in 1 mL of DMF, 100° C., 16 h.
^{[b]}GC yields.
^{[c]}Reaction run using 0.24 mmol of potassium tert-butoxide (KO^{t}Bu).
^{[d]}Reaction run using 0.25 mmol of KO^{t}Bu.

Different types of NEC ligands a-i and different NHC/Ni ratios in the coupling of different aryl halides with thiophenol were investigated (Table 3). The different types of Ni-NHC catalysts investigated were observed to be all active in this coupling reaction. Strong electron-donating NEC generated from c was observed generally to show the highest activity among NHCs a-e (Table 3). The catalytic activity was observed to be optimized at a NHC/Ni ratio of 2 (Table 3).

Bridged bidentate NHC ligands f-i were prepared, and the coupling of 4-bromotoluene with thiophenol over these catalysts was also investigated (Table 3). It was observed that with 3 mol % of nickel catalysts, the activities of catalysts with bidentate ligands were similar or slightly higher than that of (c)₂-Ni (NHC/Ni=2). However, it was observed that when 1.5 mol % of nickel catalysts was used, the activities of catalysts with f-i were ˜10 to 20% higher than that of (c)₂-Ni. No byproduct was observed over bidentate catalyst systems in contrast to ˜3 to 5% symmetric byproduct observed over (c)₂-Ni. Although the bidentate catalysts did not show significant increase in activity, they demonstrated greater stability compared to the monodentate catalysts. When more ligands were introduced in the reaction system, for instance, (c)₃-Ni or (h+c)-Ni, the catalytic activities were observed to decrease substantially. It is believed that steric hindrance from overcrowding or saturated coordination sphere of nickel center resulted in lower activities, and that further modification of the steric and electronic properties of NHC ligand to balance the catalyst stability and activity may be a consideration toward developing superior catalytic systems. Without being bound by theory, it is believed that the bidentate ligands would form more stable Ni complexes with a longer catalytic lifetime and prevent the formation of anionic or briding thiolate complexes (which might undergo slow reductive elimination as demonstrated in Pd—PR₃ systems).

TABLE 4
C—S Coupling Reactions over (c)₂-Ni(0) Catalyst^{[a]}

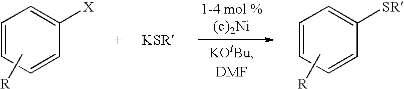

| | | catalyst | temp | | yield |
|---|---|---|---|---|---|
| entry | X | (mol %) | (° C.) | product | (%)^{[b]} |
| 1^{[d]} | I | 1 | 100 | 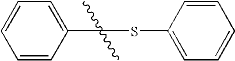 | 99 |
| 2^{[c]} | I | 1.5 | 100 | 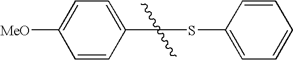 | 95 |
| 3^{[d]} | Br | 3 | 110 | 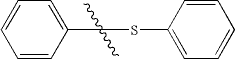 | 99 |
| 4^{[c]} | Br | 3 | 110 | 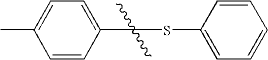 | 94 |
| 5^{[c]} | Br | 3 | 110 | 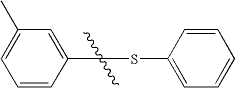 | 93 |
| 6^{[c]} | Br | 3 | 100 | 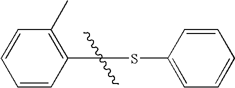 | 80 |
| 7^{[c]} | Br | 4 | 110 | 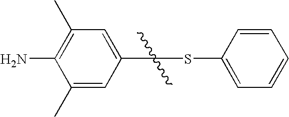 | 96 |
| 8^{[c]} | Br | 3 | 100 | 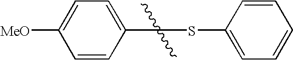 | 89 |
| 9^{[c]} | Br | 3 | 100 | 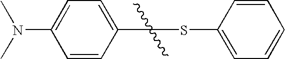 | 90 |
| 10^{[c]} | Br | 3 | 100 | 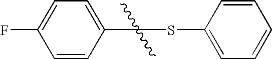 | 91 |
| 11^{[c]} | Br | 3 | 100 | 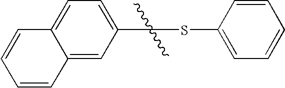 | 94 |
| 12^{[c]} | Br | 3 | 110 | 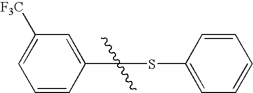 | 87 |
| 13^{[c]} | Br | 1.5 | 100 | 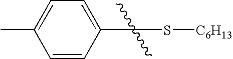 | 78 |

^{[a]}Unless otherwise specified, the reaction conditions are 1 mmol of aryl halides, 1.05 mmol of thiols, 1.1 mmol of KO^{t}Bu in 5 mL of DMF, 16 h.
^{[b]}Isolated yields.
^{[c]}Deactivated aryl halide was used as the substrate.
^{[d]}Non-activated aryl halide was used as the substrate.

Different substrates were investigated over (c)₂-Ni catalyst Excellent activities for deactivated aryl iodides were observed. Quantitative yields were observed by using 1-1.5 mol % of Ni catalyst in DMF at 80° C. for thiophenol (Table 4, entries 1-2). For electron-rich aryl bromides, high activities were observed for (c)₂-Ni. Low conversions and byproducts were observed for reactions of thiophenol with weaker bases (e.g., carbonate or phosphate). Conversions of less than 50% are considered as low conversion. When KO^{t}BU (or NaO^{t}Bu) was used as the base, good to excellent yields were observed for various substrates with 3-4 mol % of Ni catalyst (Table 4, entries 3-12). Good yield was also observed with alkyl thiol (Table 4, entry 13).

TABLE 5
C—S Coupling of Electron-Poor Aryl Halides^{[a]}

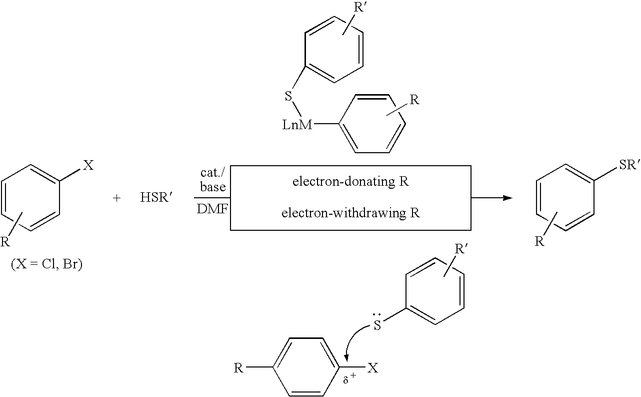

| | | catalyst | | temp | | time | Yield |
|---|---|---|---|---|---|---|---|
| Entry | X | (mol %)^{[c]} | product | (° C.) | base | (h) | (%)^{[b]} |
| 1^{[d]} | Cl | Pd- xantphos (5) | 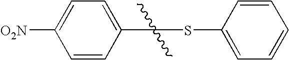 | >100 | Cs₂CO₃ | 15 | 85 |
| 2 | Cl | — | 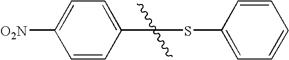 | 80 | Na₂CO₃ | 1 | 98 |
| 3 | Cl | — | 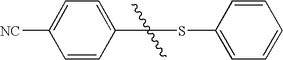 | 80 | Na₂CO₃ | 1 | 97 |
| 4 | Cl | — | 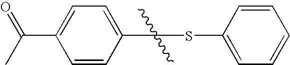 | 80 | NaO^{t}Bu | 15 | 94 |
| 5 | Cl | — | 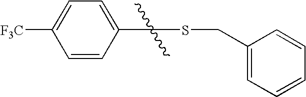 | 80 | KO^{t}Bu | 4 | 97 |
| 6 | Cl | — | 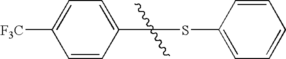 | 100 | KO^{t}Bu | 16 | 65 |
| 7 | Cl | c-Ni (1.5) | 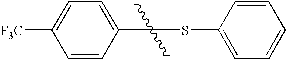 | 80 | KO^{t}Bu | 16 | 87 |
| 8 | Br | — | 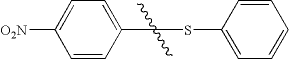 | 80 | Cs₂CO₃ | 1 | 96 |
| 9 | Br | — | 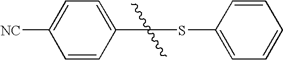 | 80 | NaO^{t}Bu | 1 | 95 |
| 10 | Br | — | 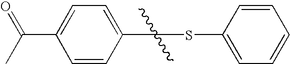 | 80 | NaO^{t}Bu | 6 | 97 |
| 11 | Br | — | 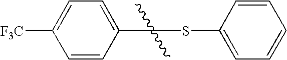 | 100 | KO^{t}Bu | 16 | 95 |
| 12 | Br | — | 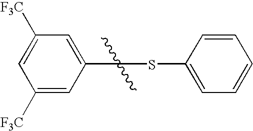 | 100 | KO^{t}Bu | 16 | 94 |
| 13 | Br | — | 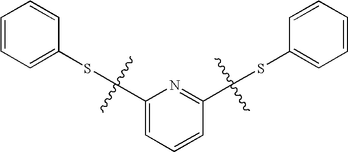 | 100 | KO^{t}Bu | 16 | 95 |
| 14 | Br | — | 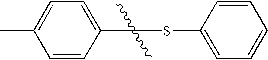 | 100 | NaO^{t}Bu | 16 | 0 |

^{[a]}Unless otherwise specified, the reaction conditions are 1 mmol of aryl halides, 1.05 mmol of thiols, 1.1 mmol of KO^{t}Bu in 5 mL of DMF.
^{[b]}Isolated yields.
^{[c]}No catalyst was used in entries 2-6, 8-14.
^{[d]}Comparative catalyst (see Itoh, T, Mase, T. Org. Lett. 2004, 6, 4587).

Although it is known that activated aryl chlorides, such as p-nitrile chlorobenzene, can follow the nucleophilic substitution mechanism to form a C—S coupling product and do not need a catalyst, the competition between nucleophilic substitution and metal-catalyzed reductive elimination pathways to certain substrates remains unclear. It has been reported that metal complexes catalyzed coupling of electron-poor aryl halides with thiols. However, it was observed that control reactions between these aryl halides with thiols also gave good to quantitative yields of C—S coupling products under similar reaction conditions (Table 5). Under these reaction conditions, the rate of nucleophilic substitution pathway on most electron-poor sp² carbon was observed to be competitive with or higher than that of metal-catalyzed reductive elimination pathway. As shown in Table 5, reactions between 1-chloro(bromo)-4-nitrobenzene or 4-chloro(bromo)benzonitrile with thiols were observed to give quantitative thioether in 1 h under relatively mild conditions (entries 1-3 and 8-9), which is different from the reported literature. Reactions between 4-chloro(bromo)acetophenone, 2,6-dibromopyridine, and 3,5-bis(trifluoromethyl)bromobenzene with thiophenol also gave quantitative yields in 8-16 h with a strong base. 4-Chloro(bromo)benzotrifluoride with thiophenol showed competitive reaction rates by two different reaction pathways. The reaction between 4-chlorobenzotrifluoride and benzylthiol with base was observed to be much faster (Table 5, entry 5). Without metal catalysts, no desired products were observed for reactions between electron-rich chloro(bromo)arenes with thiols (Table 5, entry 14).

TABLE 6
C—O coupling reactions over Ni—NHC catalysts.^{[a]}

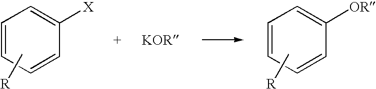

| | NHC | | Ni | | | Yield |
|---|---|---|---|---|---|---|
| Entry | NHC/Ni ratio | X | [mol %] | R | Product | [%]^{[b]} |
| 1 | a 1 | Cl | 1.5 | CF₃ | 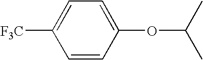 | 10 |
| 2 | c 1 | Cl | 1.5 | CF₃ | 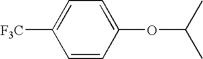 | 45 |
| 3 | c 2 | Cl | 1.5 | CF₃ | 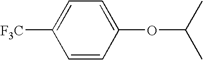 | 65 |
| 4 | c 2 | Cl | 1.5 | NO₂ | 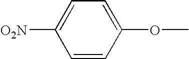 | 98 |
| 5 | c 2 | Cl | 1.5 | NO₂ | 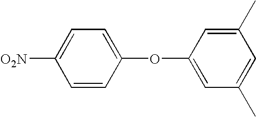 | 99 |
| 6 | c 2 | Cl | 1.5 | NO₂ | 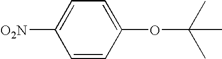 | 99 |
| 7 | e 2 | Cl | 0.1 | CN | 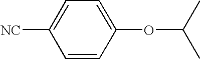 | 76 |
| 8 | e 2 | Cl | 0.1 | CN | 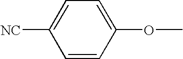 | 99 |
| 9 | e 2 | Cl | 0.1 | CN | 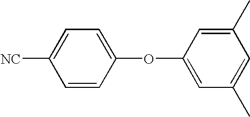 | 68 |

^{[a]}Reaction conditions: 0.2 mmol of aryl halides, 0.22 mmol of alkoxides in 1 ml of DMF, 100° C., 16 h.
^{[b]}GC yields.

Homogeneous C—O coupling reactions catalyzed by Ni-NHC complexes were investigated. As with the C—S coupling reactions, the activities of the catalysts were observed to be dependent on the type of ligands and the ligand/Ni ratio (Table 6). The activity of the Ni-NHC catalysts was observed to decrease in the following order: Ni-c (ligand/Ni ratio=2)>Ni-e (ligand/Ni ratio=2)>Ni-C (ligand/Ni ratio=1) Ni-a (ligand/Ni ratio=1). The homogeneous Ni-c catalysts were observed to have higher activities than the heterogeneous system. It is well known that the bulky NHC ligand in Pd-NHC catalyst is very important for achieving high activity in Suzuki coupling reactions. However, stereo effect was not obvious in the Ni-NHC catalysts. It was observed that electronic effect appeared to be more important for improving the catalyst performance. Ni-(c)₂ and Ni-(e)₂ showed excellent activities towards the coupling of aryl halides with alkoxides and aryloxides.

The present invention includes isomers such as geometrical isomers, optical isomers based on asymmetric carbon, stereoisomers and tautomers and is not limited by the description of the formula illustrated for the sake of convenience.

Although the foregoing invention has been described in some detail by way of illustration and example, and with regard to one or more embodiments, for the purposes of clarity of understanding, it is readily apparent to those of ordinary skill in the art in light of the teachings of this invention that certain changes, variations and modifications may be made thereto without departing from the spirit or scope of the invention as described in the appended claims.

It must be noted that as used in the specification and the appended claims, the singular forms of “a”, “an” and “the” include plural reference unless the context clearly indicates otherwise.

Unless defined otherwise all technical and scientific terms used herein have the same meaning as commonly understood to one of ordinary skill in the art to which this invention belongs.

All publications, patents and patent applications cited in this specification are incorporated herein by reference as if each individual publication, patent or patent application were specifically and individually indicated to be incorporated by reference. The citation of any publication, patent or patent application in this specification is not an admission that the publication, patent or patent application is prior art.

1. A method for carbon-sulfur (C—S) or carbon-oxygen (C—O) coupling comprising:
a) mixing, in any order, a thiol-containing compound, an aryl halide and a transition metal complex to obtain C—S coupling; or
b) mixing, in any order, an alkoxide or aryloxide, an aryl halide and a transition metal complex to obtain C—O coupling,
wherein the transition metal complex comprises a heterocyclic carbene ligand complexed with a transition metal other than palladium.

2. The method according to claim 1, wherein the heterocyclic carbene ligand is a poly-N-heterocyclic carbene (p-NHC).

3. The method according to claim 1 or 2, wherein the transition metal complex comprises a monomer unit represented by the formula (I): 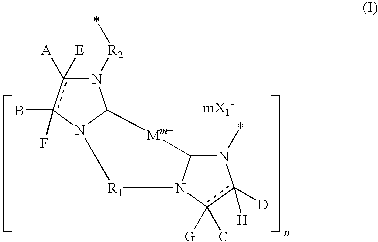
wherein:
* indicates an end of the monomer unit;
each of R₁ and R₂ is a linker group;
X₁⁻ is a counterion;
M is a transition metal;
m is an integer of 1, 2, 3, 4, 5, 6 or 7;
n is between about 5 and 1000; and [symbol]
represents a single bond or a double bond,
wherein when [symbol] represents a single bond, each of A, B, C, D, E, F, G and H is independently hydrogen or an optionally substituted substituent which is not hydrogen; any two of A, B, C, D, E, F, G and H are joined to form a cyclic structure; or any pair of substituents A, B, C, D, E, F, G and H attached to the same carbon atom represents a single substituent attached to the carbon atom by a double bond, and
wherein when [symbol] represents a double bond, E, F, G, and H are absent, and each of A, B, C and D is independently hydrogen or an optionally substituted substituent which is not hydrogen; any two of A, B, C and D are joined to form a cyclic structure; or at least one heterocyclic ring of formula (I) is fused with an aromatic or heteroaromatic ring.

4. The method according to any one of claims 1 to 3, wherein the transition metal complex is a) in the form of one or more particles, b) a heterogeneous catalyst or c) in the form of one or more particles and is a heterogeneous catalyst.

5. The method according to any one of claims 1 to 4, wherein the heterocyclic carbene ligand is poly-imidazolidene or poly-benzoimidazolidene.

6. The method according to claim 1, wherein the heterocyclic carbene ligand is a N-heterocyclic carbene (NHC).

7. The method according to claim 1 or 6, wherein the heterocyclic carbene ligand is represented by the formula (III) or (V): 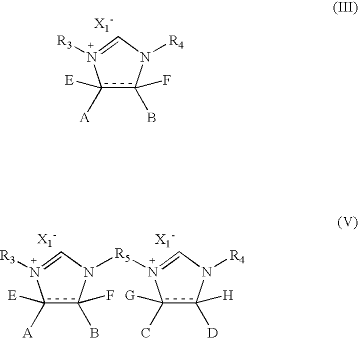
wherein in formula (III):
X₁⁻ is as defined in claim 3; [symbol]
represents a single bond or a double bond; and
each of R₃ and R₄ is independently an optionally substituted substituent which is not hydrogen,
wherein when [symbol] represents a single bond, each of A, B, E and F is independently hydrogen or an optionally substituted substituent which is not hydrogen; any two of A, B, E and F are joined to form a cyclic structure; or any pair of substitutents A, B, E, and F attached to the same carbon atom represents a single substituent attached to the carbon atom by a double bond, and
wherein when [symbol] represents a double bond, E and F are absent, and each of A and B is independently hydrogen or an optionally substituted substituent which is not hydrogen; A and B are joined to form a cyclic structure; or the heterocyclic ring of formula (III) is fused with an aromatic or heteroaromatic ring, and
wherein in formula (V):
X₁⁻ is as defined in claim 3, and R₃ and R₄ are as defined above; [symbol]
represents a single or double bond; and
R₅ is a linker group,
wherein when [symbol] represents a single bond, each of A, B, C, D, E, F, G and H is independently hydrogen or an optionally substituted substituent which is not hydrogen; any two of A, B, C, D, E, F, G and H are joined to form a cyclic structure; or any pair of substituents A, B, C, D, E, F, G and H attached to the same carbon atom represents a single substituent attached to the carbon atom by a double bond, and
wherein when [symbol] represents a double bond, E, F, G, and H are absent, and each of A, B, C and D is independently hydrogen or an optionally substituted substituent which is not hydrogen; any two of A, B, C and D are joined to form a cyclic structure; or at least one heterocyclic ring of formula (V) is fused with an aromatic or heteroaromatic ring.

8. The method according to claim 1 or 6, wherein the heterocyclic carbene ligand is represented by the formula:

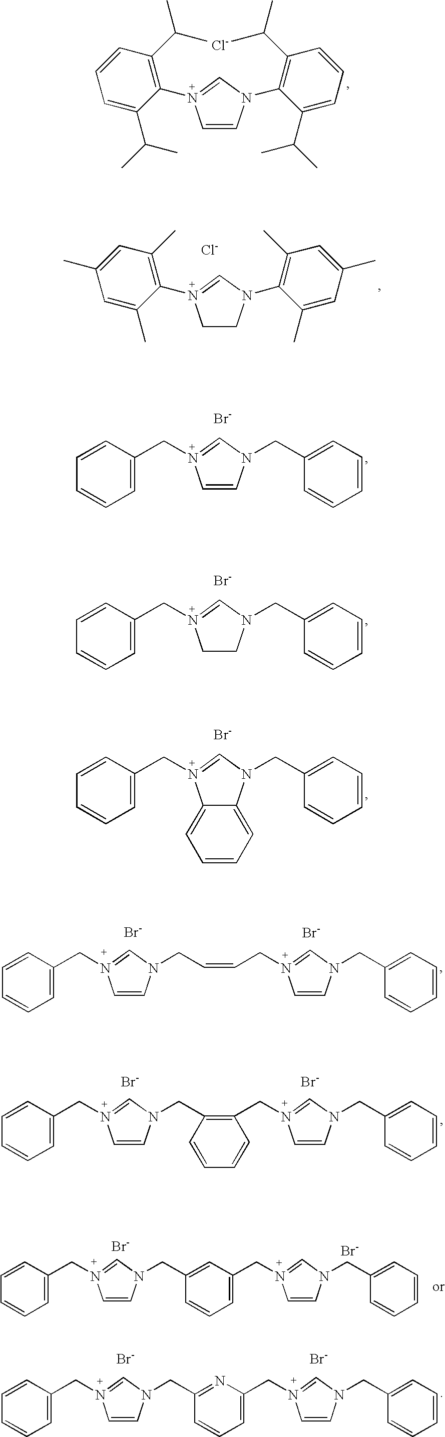

9. A method for carbon-sulfur (C—S) or carbon-oxygen (C—O) coupling comprising:
a) mixing, in any order, a thiol-containing compound, an aryl halide and a transition metal complex to obtain C—S coupling; or
b) mixing, in any order, an alkoxide or aryloxide, an aryl halide and a transition metal complex to obtain C—O coupling,
wherein the transition metal complex comprises a heterocyclic carbene ligand complexed with nickel.

10. The method according to claim 9, wherein the heterocyclic carbene ligand is a poly-N-heterocyclic carbene (p-NHC).

11. The method according to claim 9 or 10, wherein the transition metal complex comprises a monomer unit represented by the formula (I): 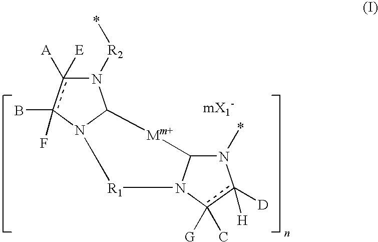
wherein:
* indicates an end of the monomer unit;
each of R₁ and R₂ is a linker group;
X₁⁻ is a counterion;
M is nickel;
m is an integer of 1, 2, 3, 4, 5, 6 or 7;
n is between about 5 and 1000; and [symbol]
represents a single bond or a double bond,
wherein when [symbol] represents a single bond, each of A, B, C, D, E, F, G and H is independently hydrogen or an optionally substituted substituent which is not hydrogen; any two of A, B, C, D, E, F, G and H are joined to form a cyclic structure; or any pair of substituents A, B, C, D, E, F, G and H attached to the same carbon atom represents a single substituent attached to the carbon atom by a double bond, and
wherein when [symbol] represents a double bond, E, F, G, and H are absent, and each of A, B, C and D is independently hydrogen or an optionally substituted substituent which is not hydrogen; any two of A, B, C and D are joined to form a cyclic structure; or at least one heterocyclic ring of formula (I) is fused with an aromatic or heteroaromatic ring.

12. The method according to any one of claims 9 to 11, wherein the transition metal complex is a) in the form of one or more particles, b) a heterogeneous catalyst or c) in the form of one or more particles and is a heterogenous catalyst.

13. The method according to any one of claims 9 to 12, wherein the transition metal complex is nickel poly-imidazolidene or nickel poly-benzoimidazolidene.

14. The method according to claim 9, wherein the heterocyclic carbene ligand is a N-heterocyclic carbene (NEC).

15. The method according to claim 9 or 14, wherein the heterocyclic carbene ligand is represented by the formula (III) or (V): 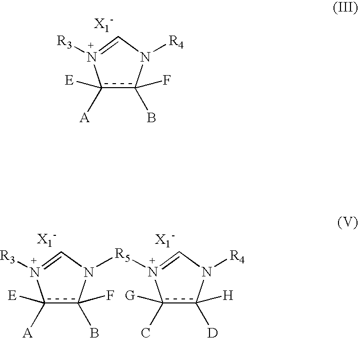
wherein in formula (III):
X₁⁻ is as defined in claim 3; [symbol]
represents a single bond or a double bond; and
each of R₃ and R₄ is independently an optionally substituted substituent which is not hydrogen,
wherein when [symbol] represents a single bond, each of A, B, E and F is independently hydrogen or an optionally substituted substituent which is not hydrogen; any two of A, B, E and F are joined to form a cyclic structure; or any pair of substitutents A, B, E, and F attached to the same carbon atom represents a single substituent attached to the carbon atom by a double bond, and
wherein when [symbol] represents a double bond, E and F are absent, and each of A and B is independently hydrogen or an optionally substituted substituent which is not hydrogen; A and B are joined to form a cyclic structure; or the heterocyclic ring of formula (III) is fused with an aromatic or heteroaromatic ring, and
wherein in formula (V):
X₁⁻ is as defined in claim 3, and R₃ and R₄ are as defined above; [symbol]
represents a single or double bond; and
R₅ is a linker group,
wherein when [symbol] represents a single bond, each of A, B, C, D, E, F, G and H is independently hydrogen or an optionally substituted substituent which is not hydrogen; any two of A, B, C, D, E, F, G and H are joined to form a cyclic structure; or any pair of substituents A, B, C, D, E, F, G and H attached to the same carbon atom represents a single substituent attached to the carbon atom by a double bond, and
wherein when [symbol] represents a double bond, E, F, G, and H are absent, and each of A, B, C and D is independently hydrogen or an optionally substituted substituent which is not hydrogen; any two of A, B, C and D are joined to form a cyclic structure; or at least one heterocyclic ring of formula (V) is fused with an aromatic or heteroaromatic ring.

16. The method according to claim 9 or 14, wherein the heterocyclic carbene ligand is represented by the formula:

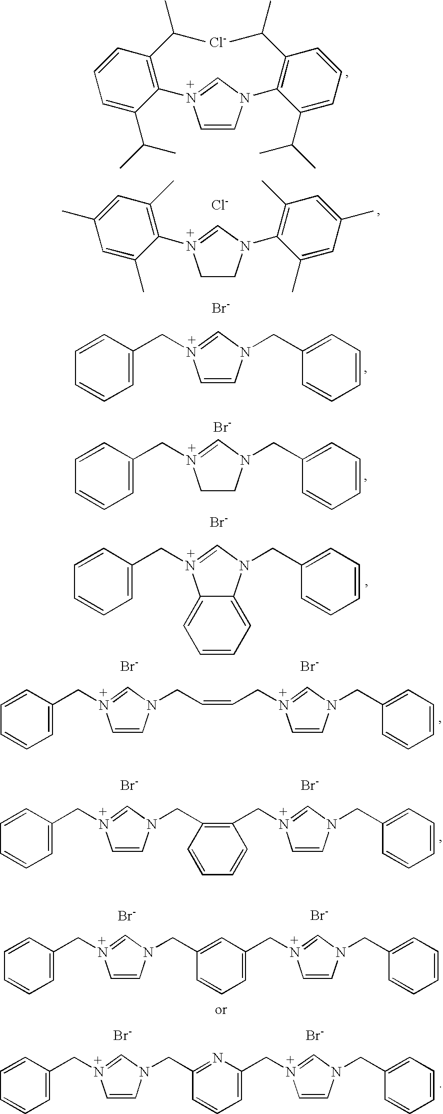

17. A transition metal complex comprising a poly-N-heterocyclic carbene (p-NHC) complexed with nickel.

18. The transition metal complex according to claim 17, which is nickel poly-imidazolidene or nickel poly-benzoimidazolidene.

19. A transition metal complex comprising a heterocyclic carbene ligand represented by the formula: 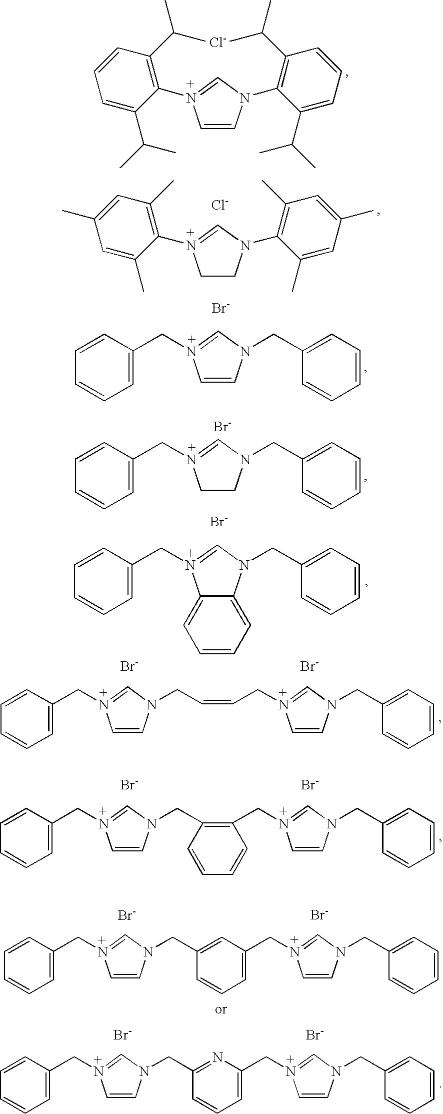
complexed with nickel.
